(19) United States
(12) Patent Application Publication
(10) Pub. No.: US 2011/0295095 A1
(43) Pub. Date: Dec. 1, 2011

(54) Meter with bi-stable display

(76) Inventors: Michael Deppert, Bensheim (DE); Dietmar Stadler, Queidersbach (DE); Guenter Handwerker, Boehl-lggelheim (DE); Stefan Lindegger, Huttwil (CH); Kurt Friedli, Lyssach (CH); Dieter Hofmann, Halle (DE); Kristin Mueckel, Halle (DE)

(21) Appl. No.: 12/955,467

(22) Filed: Nov. 29, 2010

Related U.S. Application Data
(63) Continuation of application No. PCT/EP2009/002950, filed on Apr. 23, 2009.

(30) Foreign Application Priority Data
May 31, 2008 (EP) 08010004.3

(51) Int. Cl. A61B 5/145; A61M 37/00; A61M 1/00; A61B 5/00

(52) U.S. Cl. 600/365; 600/300; 604/19; 600/309; 604/151

(57) ABSTRACT

A medical handheld device having a bi-stable display, which can be supplemented by a further display, is presented. The display of the medical handheld device can have a display surface. The display surface can have a keypad in the form of a matrix of sensors. The matrix of sensors can be coupled to image segments on the display surface and can detect touches on the display surface. By coupling the matrix of sensors to the image segments of the display surface, a change of the representation of touched areas of the display surface can occur.

### CROSS-REFERENCE TO RELATED APPLICATIONS

This application is a continuation of PCT/EP2009/002950, filed Apr. 23, 2009, which is based on and claims priority to EP 08010004.3, filed May 31, 2008, which is hereby incorporated by reference.

### BACKGROUND

The present disclosure generally relates to a medical handheld device for administering insulin or for determining the blood glucose concentration and, in particular, to a medical handheld device for administering insulin or for determining the blood glucose concentration comprising an electrically switchable display unit.

Typically, liquid crystal displays are used as electrically switchable displays for medical handheld devices to display important health data, such as, for example, analyte concentrations in bodily fluids or the time, or dose, of the delivery of a medical agent. Corresponding information can be accessed simply by the users of medical handheld devices of this type. Additionally, corresponding information can typically be presented in a comprehensible manner.

In order to show important information as noticeably as possible, colored and bright liquid crystal displays are frequently used in medical handheld devices. During use of such a medical handheld device, the liquid crystal display may be activated with other device functions and displays of other required information. To activate the display, a user may have to actuate individual operating elements and select the desired information from an extensive menu. These actions may be cumbersome for many users

In addition, because of the significant power consumption of such liquid crystal displays, medical handheld devices usually have an automatic shutoff function. If no actuation of an operating element occurs during a specified period of time, the liquid crystal display shuts down in order to prevent excessive power consumption. This automatic shutoff can be annoying to many users, especially for those whom it may be difficult to comprehend the displayed data in a short time.

Therefore, there is a need to access and display important data for users of medical handheld devices easily and simply.

### SUMMARY

According to the present disclosure, a medical handheld device for administering a medical agent or for measuring an analyte concentration of a body fluid is presented. The medical handheld device can comprise an electrically switchable display. The display can be a bistable display.

In accordance with one embodiment of the present disclosure, the display may have a display surface having a keypad in the form of a matrix of sensors. The matrix of sensors may be coupled to image segments of the display surface and may detect a touch on the display surface. For a medical handheld device for administering a medical agent or for measuring an analyte concentration of a body fluid, the matrix of sensors may cause a change of the representation of touched areas of the display surface through the coupling of the matrix of sensors to image segments of the display surface.

In accordance with another embodiment of the present disclosure, the medical handheld device can comprise a first electrically switchable display for displaying function-related state information of the handheld device for administering a medical agent or for measuring an analyte concentration of a body fluid and a second electrically switchable display for displaying data independent of the device function, in particular graphic data.

Accordingly, it is a feature of the embodiments of the present disclosure to access and display important data for users of medical handheld devices easily and simply through the use of bistable display. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates an exemplary embodiment of a front view of a medical handheld device according to an embodiment of the present disclosure.

FIG. 2 illustrates an exemplary embodiment of a rear view of a medical handheld device according to an embodiment of the present disclosure.

FIG. 3 illustrates the exemplary embodiment of a rear view of a medical handheld device of FIG. 2 with a note according to an embodiment of the present disclosure.

FIG. 4 illustrates the exemplary embodiment of a rear view of a medical handheld device of FIG. 2 with a decorative motif according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

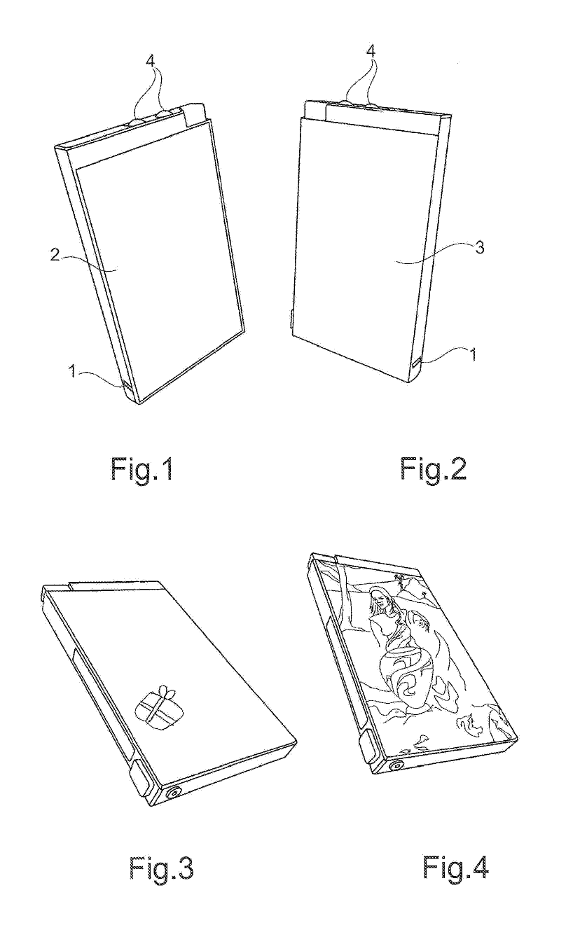

Referring initially to FIG. 1, a medical handheld device is illustrated. The medical handheld device can be a measuring device for determining an analyte concentration of a bodily fluid such as, for example, the blood glucose concentration. The measuring device can operate using test strips (not shown), to which a sample of a bodily fluid, typically blood and/or interstitial liquid, is applied to be assayed. The test strips may be stored in a magazine in the interior of the measuring device and exposed through a device opening 1 when needed, so that a sample can be applied to the test strip.

In one embodiment, test strips of this type are typical for blood sugar measuring devices and may have test areas on the strips with detection reagents. The detection regents can display a coloration as a function of the analyte concentration within the liquid sample which can be determined upon contact with a liquid sample. This coloration can then be photometrically analyzed in the measuring device. In another embodiment, test strips for the electrochemical detection of the analyte concentration can be utilized.

As illustrated in FIGS. 1 and 2, the handheld device can have a first electrically switchable display 2 on its front side and a second electrically switchable display 3 on its rear side. In one embodiment, the first electrically switchable display 2 and the second electrically switchable display 3 can be implemented as bistable liquid crystal displays. In contrast to a typical liquid crystal displays, the liquid crystal of a bistable liquid crystal display can have not only one, but two stable states. A transition of the liquid crystal between the two stable states can be caused by an electrical field. If the liquid crystal of a bistable display has assumed one of its two stable states, it can remain in this state until a state change is induced by an electrical field. Alternatively, in another embodiment, bistable display units may also be implemented as electrophoretic systems.

Bistable displays can have the advantage that power may only be required upon a change of the display, i.e., a change of image segments. A display once set, i.e., image segments or pixels switched to visible, can maintain its state. In a medical handheld device with a bistable display unit, information may therefore be displayed for a long time without power consumption. Even when the medical handheld device is shut off, information may remain displayed. Therefore, in one embodiment, information about a sequence of measurement results can advantageously be displayed, so that a user can readily be reminded again and again of important results as well as reminded of the effectiveness of a medical treatment, such as insulin doses or maintaining a diet, can be displayed to the user. For example, the time curve of the glucose concentration of a bodily fluid can be shown in a graph on the bistable display.

In one embodiment, the first liquid crystal display 2 on the front side of the medical handheld device can shut off automatically after a predefined time has passed since the last input. In this manner, a power source for the medical handheld device may not be excessively strained. In one example embodiment, the power source can be positioned within the interior of the medical handheld device. In one embodiment, the power source can be a battery but any other suitable source of power may be used. Therefore, one important advantage of the medical handheld device can be that important information can be accessible even after a failure of the medical handheld device or its power supply.

The first liquid crystal display 2 can have a light source, so that it can light up during operation and, therefore, the first liquid crystal display 2 may also be read in the dark. In one embodiment, the first liquid crystal display 2 on the front side can be an illuminated, colored liquid crystal display. Such a display can be used during the operation of the medical handheld device for displaying device information and/or measurement results.

In one example embodiment, the first liquid crystal display 2 can be equipped with touch sensors. These touch sensors can form operating elements of the medical handheld device such as, for example, a sensor display screen or touch screen. By touching the display surface of the first liquid crystal display 2, a user can operate the medical handheld device and can input instructions.

In one embodiment, the second electrically switchable display 3 can be a bistable liquid crystal display. This second electrically switchable display 3 can be positioned on the rear side of the medical handheld device. As mentioned above, bistable liquid crystal displays can have the advantage that an electrical field, and thus power consumption, is only required upon a change of the image. A display on the second electrically switchable display 3 on the rear side of the medical handheld device can therefore be maintained for a long time.

In another embodiment, the second electrically switchable display 3 can be a monostable display. Monostable displays are displays which have one stable state, typically the dark state, so that power may be required to maintain a display. Common liquid crystal displays are monostable displays. A monostable display can advantageously have a light source, using which can illuminate the display in poor light conditions. In one embodiment, this light source can be implemented by self-lighting image segments, for example, in an LED or OLED display. In another embodiment, this light source can be implemented independently of the image segments, such as, for example, as a background light which can be turned on as needed. In one embodiment, a separate light source that can be turned on as needed may also be added to a bistable display unit.

The use of a monostable display having self-lighting image segments may have the advantages of the representation of information by a bright, and thus energy-intensive, colored liquid crystal display combined with those of an energy-efficient bistable display. In one example embodiment, if the medical handheld device is activated for a measurement or administering an agent dose for a briefer or longer time, a user can read information from the lighted liquid crystal display with high user comfort. During the remaining time, important information can be available via the bistable display unit. The lighted liquid crystal display can be equipped with an automatic shutoff function, so that the display can automatically shut off if there no actuation after an established time span.

The current operating parameters of the medical handheld device, such as, for example, the current delivery rate of an agent dose, can be displayed using the monostable display, in order, in the event of a device defect, to prevent correct operation from being simulated in spite of a defect, due to a display of the bistable display unit which may no longer be relevant. If this information is displayed on the bistable display unit, the information may be include a time of day, for example, so that obsolete information can always recognized as such.

In one embodiment, the second bistable display 3 can advantageously be used for displaying information of the last measurement, such as, for example, date and measurement result or for displaying information on a sequence of measurements, such as, for example, the curve of the glucose concentration. Therefore, this information may be read even when the medical handheld device is shut down.

In medical handheld devices for delivering medical agents, such as, for example, insulin, the time and dose of the last agent dose and/or an indication of the next required dose may be displayed using a bistable display. In the case of a medical handheld device for continuous or quasi-continuous agent dosing, such as, for example, an insulin pump, the delivery rate and/or the fill level of a medication reservoir can also be displayed. In the case of combined medical handheld devices, which integrate a measuring device and a delivery device, a plurality, or all, of the information concerning these devices may also be displayed at the same time. In addition, information about the charge state of its power source can also be displayed in medical handheld devices having the bistable display unit

Important medical information which may be continuously displayed on the medical handheld device can be, for example, data of the last measurement, such as, for example, time, date, and measurement result; information on a sequence of measurements, such as, for example, the progression-curve of the glucose concentration; information about measurements that may be supplemented by a symbol indicating whether the measurement was performed before or after a meal; therapy recommendations; time of the next measurement or agent dosing to be performed; time of the last agent dose; agent dosing rate; time of occurrence of a device defect; and combinations thereof.

In one embodiment, telephone numbers for emergencies or other emergency instructions, device information, user name, and possibly address or the name of an attending physician having contact information can be displayed on the medical handheld device. In one example embodiment, if the medical handheld device is an insulin pump for continuous or quasi-continuous insulin dosing, information about performed insulin doses can be of special significance. This type of information may be displayed using the bistable display unit, even in the case of a device failure. The hazards connected to a device failure may thus be significantly reduced, because the information regarding administered agent doses may not be lost.

In one embodiment, in the event of a device failure, the source of the possible malfunction can be displayed, for example, in the form of an error code. Additionally, before the medical handheld device is first put into operation, the bistable display can display instructions for operation or any other suitable device information such as product or serial number. In other embodiments, the bistable display can display information regarding the production process, such as, for example, on a testing status, or a barcode.

In one embodiment, the second bistable display 3 can have a display surface that can be implemented as a keypad. The keyboard can be in the form of a matrix of sensors that can detect touch. In this embodiment, the display surface can be used as an operating element. Display surfaces having such a keyboard are frequently called touch screens. The matrix of sensors can be coupled to image segments on the display surface and can cause a change of the representation, such as, for example, the brightness or color, of touched areas of the display surface. The display surface can then be employed by a user as a writing surface for notes. In one embodiment, the display surface of the second bistable display 3 can be used as a notepad, on which the user can draw sketches or record handwritten notes, such as telephone numbers. In one embodiment, the sensors can be pressure sensitive, so that handwritten notes or drawings may be inputted using a finger or a stylus, which can then be displayed on the display surface of the second bistable display 3. FIG. 3 shows an example of such a note. This note can be displayed for a long time without power consumption and can be erased at any time, as desired by a suitable input command. In another embodiment, the second bistable display 3 can be “e-paper” and may be implemented, for example, as bistable liquid crystal displays or electrophoretic displays.

In one embodiment, the second bistable display 3 can additionally also be used for decorating the handheld device with an image selectable by a user as illustrated in FIG. 4. A selection of suitable motifs, images, or patterns can be stored by the processor in a memory of the medical handheld device, for example. In one embodiment, these images may be used, for example, the background of the second bistable display 3.

In one example embodiment, the medical handheld device can have a first electrically switchable display 2 for displaying function-related state information of the medical handheld device and a second electrically switchable display 3 for displaying data that may independent of the device function, such as, for example graphical data. Function-related state information can be displayed during operation of the medical handheld device, such as, for example, a reaction to control input of a user, and can be, for example, specifications of the medical handheld device, a measurement was taken, or a measurement result. Data independent of the medical handheld device function can be, for example, data that is not related to medical functions or functions of the medical handheld device. Examples of medical functions can be the measurement of medically significant parameters, such as, for example, analyte concentrations of bodily fluids, or the administration of agents, such as, for example, insulin. Examples of data being independent of device function can be notes of a user or a decorative graphic. In one embodiment, the second electrically switchable display 3 of such a handheld device can be a bistable display unit.

The medical handheld device can also output medical data, such as, for example, measurement results, via the interface 4.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A medical handheld device, the medical handheld device comprising,
an electrically switchable display, wherein the electrically switchable display is a bistable display unit.

2. The medical handheld device according to claim 1, further comprising,
a second electrically switchable display.

3. The medical handheld device according to claim 2, wherein the second electrically switchable display is a monostable display.

4. The medical handheld device according to claim 1, wherein the medical handheld device administers a medical agent.

5. The medical handheld device according to claim 4, wherein the medical agent is insulin.

6. The medical handheld device according to claim 5, wherein the bistable display displays the time of day of the last insulin dose.

7. The medical handheld device according to claim 6, wherein the bistable display displays the dose, insulin dosing rate, or combinations thereof.

8. The medical handheld device according to claim 4, further comprises,
a pump for the administration of a medical agent.

9. The medical handheld device according to claim 1, wherein the medical handheld device measures an analyte concentration of a body fluid.

10. The medical handheld device according to claim 8, wherein the medical handheld device measures blood sugar concentration.

11. The medical handheld device according to claim 8, wherein the medical handheld device displays a measurement result using the bistable display when the medical handheld device is shut down.

12. The medical handheld device according to claim 8, wherein the medical handheld device displays information about a sequence of measurement results using the bistable display when the device is shut down.

13. The medical handheld device according to claim 1, wherein before the medical handheld device is put into operation, the medical handheld device displays instructions for operation using the bistable display.

14. The medical handheld device according to claim 1, wherein the bistable display displays emergency instructions.

15. The medical handheld device according to claim 14, wherein the displayed emergency instructions is a telephone number.

16. The medical handheld device according to claim 1, wherein the bistable display displays file contents.

17. The medical handheld device according to claim 16, wherein the displayed file contents are graphic files.

18. The medical handheld device according to claim 1, wherein the bistable display comprises,
a display surface implemented as a keypad, wherein the keyboard comprises a matrix of sensors coupled to image segments of the display surface which cause a change of representation of touched areas of the display surface.

19. The medical handheld device according to claim 18, wherein the display surface is a writing surface for notes

20. A display for a medical handheld device for administering a medical agent or for measuring an analyte concentration of a body fluid, the display comprising:
a display surface having a keypad in the form of a matrix of sensors coupled to image segments of the display surface that detect a touch of the display surface, the matrix of sensors cause a change of the representation of touched areas of the display surface through the coupling to image segments of the display surface.

21. A medical handheld device for administering a medical agent or for measuring an analyte concentration of a body fluid, the medical handheld device comprising:
a first electrically switchable display for displaying function-related state information of the medical handheld device; and
a second electrically switchable display for displaying data independent of the device function.

22. The medical handheld device according to claim 21, wherein the data independent of device function includes graphic data.
